(19) United States
(12) Patent Application Publication
(10) Pub. No.: US 2025/0081301 A1
(43) Pub. Date: Mar. 6, 2025

(54) MICROWAVE OVEN APPLIANCE INCLUDING AN AIR FRESHENER COMPARTMENT AND METHOD OF OPERATING THE SAME

(71) Applicant: Haier US Appliance Solutions, Inc., Wilmington, DE (US)

(72) Inventors: Bagawathkumar Chellappan, Prospect, KY (US); Mark Heimerdinger, Louisville, KY (US)

(21) Appl. No.: 18/461,052

(22) Filed: Sep. 5, 2023

(51) Int. Cl. H05B 6/64; A61L 9/12; H05B 6/66

(52) U.S. Cl. CPC H05B 6/6423; A61L 9/122; H05B 6/664; A61L 9/014

(57) ABSTRACT

A microwave oven appliance includes a cabinet, the cabinet forming a cooking cavity and a heating assembly cavity adjacent to the cooking cavity; a door rotatably coupled to the cabinet to provide selective access to the cooking cavity; a user interface panel coupled to the cabinet adjacent to the door; a freshener compartment provided behind the user interface panel along the transverse direction and recessed into the heating assembly cavity, the freshener compartment being in fluid communication with the heating assembly cavity, wherein the user interface panel is movable to provide selective access to the freshener compartment; and a heating assembly provided within the heating assembly cavity, the heating assembly including a fan to selectively urge air into the freshener compartment.

### FIELD OF THE INVENTION

The present subject matter relates generally to microwave oven appliances, and more particularly to compartments for air fresheners and methods of operating an air freshener mode of a microwave oven appliance.

### BACKGROUND OF THE INVENTION

Microwave oven appliances generally include a cabinet that defines a cooking chamber for receipt of food items for cooking. These appliances typically include one or more heating elements for generating energy to heat the food items during a cooking process. For example, microwave ovens typically include at least one source of electromagnetic radiation in the microwave frequency range, such as a cavity magnetron. In order to provide selective access to the cooking chamber and to contain food particles and cooking energy (e.g., microwaves) during a cooking operation, microwave appliances further include a door that is typically pivotally mounted to the cabinet.

Food and other items heated or cooked within the cooking chamber or cavity can produce strong odors. These odors may emanate from the appliance and linger in and around the room in which the microwave is situated (such as a kitchen or kitchenette). Users of the appliance and any patrons within the room or space may find the odors to be unpleasant. However, current methods for reducing kitchen odors have several drawbacks. For instance, separate scent releasers plugged into different outlets may reduce odors in places away from the source of the odor, resulting in less than desirable effects.

Accordingly, a microwave oven appliance that obviates one or more of the above-mentioned drawbacks would be beneficial. In particular, a microwave oven appliance with an integrated fragrance chamber would be useful.

### BRIEF DESCRIPTION OF THE INVENTION

Aspects and advantages of the invention will be set forth in part in the following description, or may be obvious from the description, or may be learned through practice of the invention.

In one exemplary aspect of the present disclosure, a microwave oven appliance is provided. The microwave oven appliance may include a cabinet, the cabinet forming a cooking cavity and a heating assembly cavity adjacent to the cooking cavity; a door rotatably coupled to the cabinet to provide selective access to the cooking cavity; a user interface panel coupled to the cabinet adjacent to the door; a freshener compartment provided behind the user interface panel along the transverse direction and recessed into the heating assembly cavity, the freshener compartment being in fluid communication with the heating assembly cavity, wherein the user interface panel is movable to provide selective access to the freshener compartment; and a heating assembly provided within the heating assembly cavity, the heating assembly including a fan to selectively urge air into the freshener compartment.

In another exemplary aspect of the present disclosure, a method of operating a microwave oven appliance is provided. The microwave oven appliance may include a cabinet, a user interface panel coupled to the cabinet, a fan provided within the cabinet, and a freshener compartment provided behind the user interface panel. The method may include obtaining an initiation signal to initiate a freshening operation; determining a first length of time for executing the freshening operation in response to obtaining the initiation signal; and directing the fan according to a first predetermined operational speed for the determined first length of time.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a front perspective view of a microwave oven appliance according to exemplary embodiments of the present disclosure.

FIG. 2 provides a perspective view of a microwave oven appliance according to another embodiment with a compartment door in an open position.

FIG. 3 provides a perspective view of a freshener compartment of the microwave oven appliance of FIG. 2 according to exemplary embodiments of the present disclosure.

FIG. 4 provides a front view of an air inlet to the freshener compartment of FIG. 3, with a choker slider in an open position.

FIG. 5 provides a front view of the air inlet to the freshener compartment of FIG. 3, with the choker slider in a choked position.

FIG. 6 provides a rear perspective view of a heating assembly cavity of the microwave oven appliance of FIG. 2, with a damper door in an open position.

FIG. 7 provides a rear perspective view of the heating assembly cavity of the microwave oven appliance of FIG. 2, with a damper door in a closed position.

FIG. 8 provides a flow chart illustrating a method of operating a microwave oven appliance according to exemplary embodiments of the present disclosure.

Repeat use of reference characters in the present specification and drawings is intended to represent the same or analogous features or elements of the present invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “first,” “second,” and “third” may be used interchangeably to distinguish one component from another and are not intended to signify location or importance of the individual components. The terms “includes” and “including” are intended to be inclusive in a manner similar to the term “comprising.” Similarly, the term “or” is generally intended to be inclusive (i.e., “A or B” is intended to mean “A or B or both”). In addition, here and throughout the specification and claims, range limitations may be combined and/or interchanged. Such ranges are identified and include all the sub-ranges contained therein unless context or language indicates otherwise. For example, all ranges disclosed herein are inclusive of the endpoints, and the endpoints are independently combinable with each other. The singular forms “a,” “an,” and “the” include plural references unless the context clearly dictates otherwise.

Approximating language, as used herein throughout the specification and claims, may be applied to modify any quantitative representation that could permissibly vary without resulting in a change in the basic function to which it is related. Accordingly, a value modified by a term or terms, such as “generally,” “about,” “approximately,” and “substantially,” are not to be limited to the precise value specified. In at least some instances, the approximating language may correspond to the precision of an instrument for measuring the value, or the precision of the methods or machines for constructing or manufacturing the components and/or systems. For example, the approximating language may refer to being within a 10 percent margin, i.e., including values within ten percent greater or less than the stated value. In this regard, for example, when used in the context of an angle or direction, such terms include within ten degrees greater or less than the stated angle or direction, e.g., “generally vertical” includes forming an angle of up to ten degrees in any direction, e.g., clockwise or counterclockwise, with the vertical direction V.

The word “exemplary” is used herein to mean “serving as an example, instance, or illustration.” In addition, references to “an embodiment” or “one embodiment” does not necessarily refer to the same embodiment, although it may. Any implementation described herein as “exemplary” or “an embodiment” is not necessarily to be construed as preferred or advantageous over other implementations. Moreover, each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

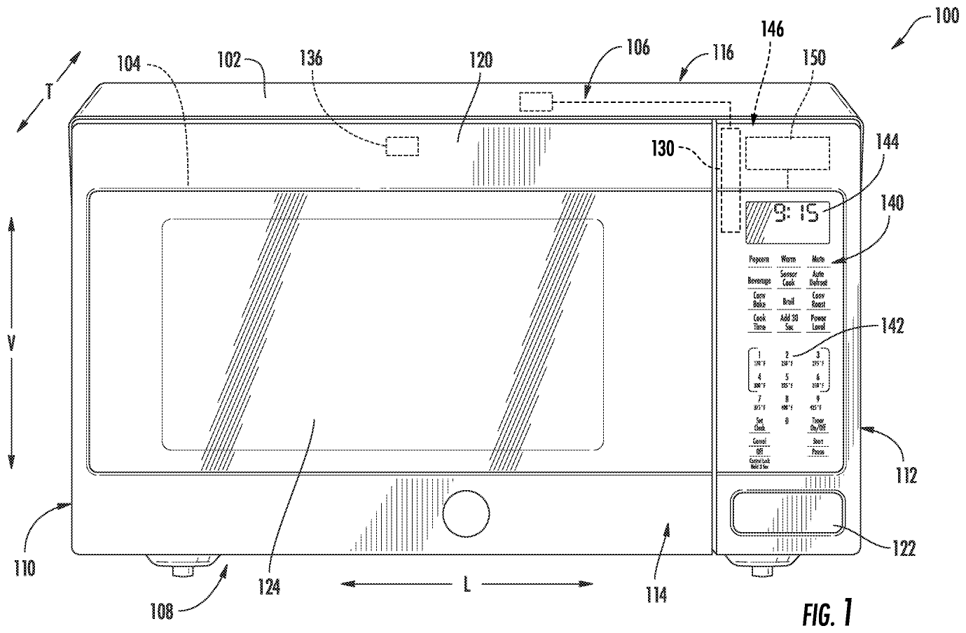

Referring now to the figures, FIG. 1 provides a front view of a microwave oven 100 as may be employed with the present subject matter. Microwave oven 100 includes an insulated cabinet 102 that defines a cooking chamber 104 for receipt of food items for cooking. As will be understood by those skilled in the art, microwave oven 100 is provided by way of example only, and the present subject matter may be used in any suitable microwave oven, such as a countertop microwave oven, an over-the-range microwave oven, etc. Thus, the example embodiment shown in the figures is not intended to limit the present subject matter to any particular cooking chamber configuration or arrangement.

As illustrated, microwave oven 100 generally defines a vertical direction V, a lateral direction L, and a transverse direction T, each of which is mutually perpendicular, such that an orthogonal coordinate system is generally defined. Cabinet 102 of microwave oven 100 extends between a top 106 and a bottom 108 along the vertical direction V, between a first side 110 (left side when viewed from front) and a second side 112 (right side when viewed from front) along the lateral direction L, and between a front 114 and a rear 116 along the transverse direction T.

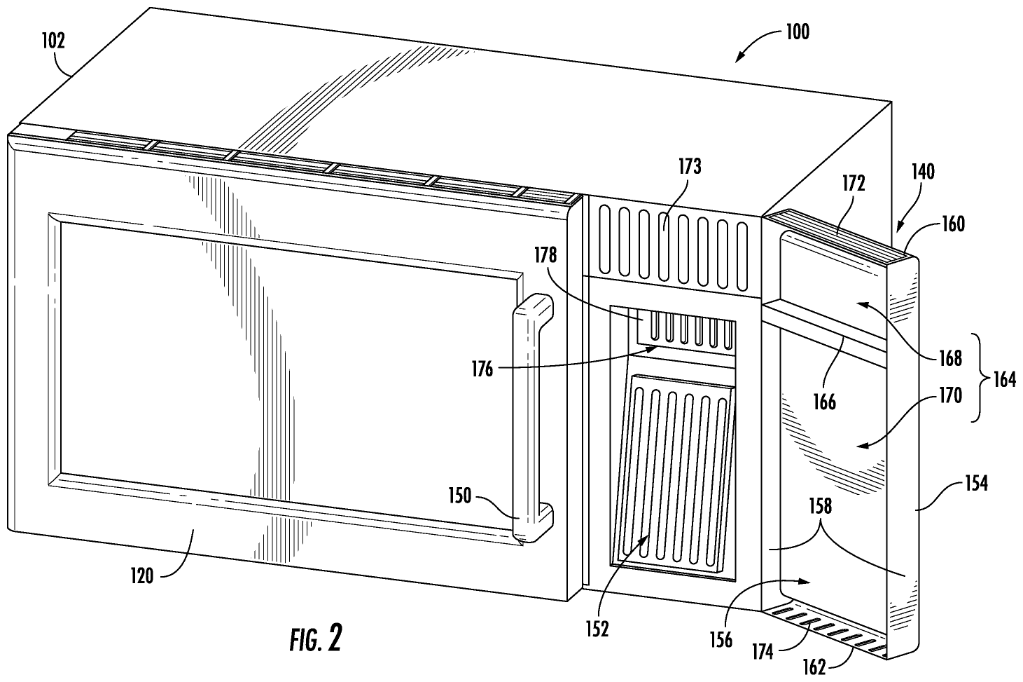

Microwave oven 100 includes a door 120 that is rotatably attached to cabinet 102 in order to permit selective access to cooking chamber 104. A handle (FIG. 2) may be mounted to door 120 to assist a user with opening and closing door 120 in order to access cooking chamber 104. As an example, a user can pull on the handle mounted to door 120 to open or close door 120 and access cooking chamber 104. Alternatively, microwave oven 100 may include a door release button 122 that disengages or otherwise pushes open door 120 when depressed. Glass windowpanes 124 provide for viewing the contents of cooking chamber 104 when door 120 is closed and also assist with insulating cooking chamber 104.

Microwave oven 100 is generally configured to heat articles, e.g., food or beverages, within cooking chamber 104 using electromagnetic radiation. Microwave appliance 100 may include various components which operate to produce the electromagnetic radiation, as is generally understood. For example, microwave appliance 100 may include a microwave heating assembly 130 which may include a magnetron (such as, for example, a cavity magnetron), a high voltage transformer, a high voltage capacitor and a high voltage diode.

According to exemplary embodiments, microwave oven 100 may further include an inverter power supply 132 that is operably coupled to microwave heating assembly 130 to provide energy from a suitable energy source (such as an electrical outlet) to microwave heating assembly 130, e.g., the magnetron. The magnetron may convert the energy to electromagnetic radiation, specifically microwave radiation. Microwave heating assembly 130 and/or inverter power supply 132 may include other suitable components, such as a capacitor that generally connects the magnetron and power supply, such as via high voltage diode, to a chassis. Microwave radiation produced by the magnetron may also be transmitted through a waveguide to cooking chamber 104.

As would be appreciated by one having ordinary skill in the art, inverter power supply 132 allows the magnetron's analog electric field intensity to be adjusted between various power levels, such as between 10% and 100% of the total power capacity. By contrast, with conventional non-inverter power supplies, the electric field intensity is either 100% or 0%, and power levels are made using a timed duty cycle. For example, a non-inverter power supply set for a 50% power level would turn the magnetron ON at 100% output power for 15 seconds, and then OFF for 15 seconds. At power levels less than 100%, inverter power supply 132 has much better heating uniformity and less penetration depth-ideal heating for sous vide as the inverter power supply heats the water while avoiding direct heating of the food with microwave energy.

The structure and intended function of microwave ovens are generally understood by those of ordinary skill in the art and are not described in further detail herein. According to alternative embodiments, microwave oven may include one or more heating elements, such as electric resistance heating elements, gas burners, other microwave heating elements, halogen heating elements, or suitable combinations thereof, are positioned within cooking chamber 104 for heating cooking chamber 104 and food items positioned therein.

Microwave oven 100 may include additional features to improve heating uniformity and precision. For example, according to an exemplary embodiment, microwave oven 100 may includes a turntable rotatably mounted within cooking chamber 104. The turntable may be selectively rotated during a cooking process to ensure improved temperature uniformity for the object being heated. In addition, microwave oven 100 may include an infrared temperature sensing array 136 that can measure temperatures across the entire bottom of the cooking chamber 104. Temperature sensing array 136 may detect temperatures at various distinct temperature locations, may associate certain locations with the food items being cooked, and may use a subset of the temperature data as feedback for regulating inverter power supply 132 and microwave heating assembly 130 for improved precision. For example, temperature sensing array 136 may include one or more infrared sensors mounted to a top of cooking chamber 104 for periodically or continuously monitoring a surface temperature of the item being heated.

A user interface panel 140 and a user input device 142 may be positioned on an exterior of the cabinet 102. The user interface panel 140 may represent a general purpose Input/Output (“GPIO”) device or functional block. In some embodiments, the user interface panel 140 may include or be in operative communication with user input device 142, such as one or more of a variety of digital, analog, electrical, mechanical or electro-mechanical input devices including rotary dials, control knobs, push buttons, and touch pads. The user input device 142 is generally positioned proximate to the user interface panel 140, and in some embodiments, the user input device 142 may be positioned on the user interface panel 140. The user interface panel 140 may include a display component 144, such as a digital or analog display device designed to provide operational feedback to a user.

Generally, microwave oven 100 may include a controller 150 in operative communication with the user input device 142. The user interface panel 140 of the microwave oven 100 may be in communication with the controller 150 via, for example, one or more signal lines or shared communication busses, and signals generated in controller 150 operate microwave oven 100 in response to user input via the user input devices 142. Input/Output (“I/O”) signals may be routed between controller 150 and various operational components of microwave oven 100. Operation of microwave oven 100 can be regulated by the controller 150 that is operatively coupled to the user interface panel 140.

Controller 150 is a “processing device” or “controller” and may be embodied as described herein. Controller 150 may include a memory and one or more microprocessors, microcontrollers, application-specific integrated circuits (ASICS), CPUs or the like, such as general or special purpose microprocessors operable to execute programming instructions or micro-control code associated with operation of microwave oven 100, and controller 150 is not restricted necessarily to a single element. The memory may represent random access memory such as DRAM, or read only memory such as ROM, electrically erasable, programmable read only memory (EEPROM), or FLASH. In one embodiment, the processor executes programming instructions stored in memory. The memory may be a separate component from the processor or may be included onboard within the processor. Alternatively, a controller 150 may be constructed without using a microprocessor, e.g., using a combination of discrete analog and/or digital logic circuitry (such as switches, amplifiers, integrators, comparators, flip-flops, AND gates, and the like) to perform control functionality instead of relying upon software.

Cabinet 102 may further form a heating assembly cavity 146 (FIG. 6). Heating assembly cavity 146 may be positioned adjacent to cooking cavity 104. Heating assembly cavity 146 may accommodate microwave heating assembly 130 therein. For instance, as described above, a magnetron may be positioned within heating assembly cavity 146 to provide microwave energy to the cooking cavity 104. Although heating assembly cavity 146 is shown as being positioned alongside cooking cavity 104 along the lateral direction L, it should be understood that a positioning of heating assembly cavity 146 may vary according to specific embodiments and applications.

Referring now to FIG. 2, a microwave oven appliance 100 according to another embodiment is provided. It should be understood that certain features may be similar to those described above with reference to FIG. 1, and accordingly, like reference numerals will be used to described like features where appropriate. As mentioned above, door 120 may include handle 150. According to this embodiment, button 122 may be omitted. Additionally or alternatively, button 122 may be incorporated to open a secondary compartment, described herein.

User interface panel 140 may be coupled to cabinet 102 adjacent to door 120. For instance, user interface panel 140 may be movable with respect to cabinet 102. According to at least some embodiments, user interface panel is coupled to cabinet 102 via one or more hinges (e.g., rotatable hinges, as would be understood) so as to rotate away from cabinet 102. As shown in FIG. 2, for example, user interface panel 140 rotates opposite door 120. For instance, when door 120 rotates outward toward first side 110 of cabinet 102, user interface panel 140 rotates outward toward second side 112 of cabinet 102.

A freshener compartment 152 may be formed into cabinet 102. Freshener compartment 152 may be positioned behind user interface panel 140 (e.g., along the transverse direction T). For instance, freshener compartment 152 may be positioned adjacent to cooking cavity 104 along the lateral direction L. Freshener compartment 152 may be configured so as to selectively receive a scent module (e.g., an air freshener pack) therein. Thus, freshener compartment 152 may be recessed rearward along the transverse direction T into cabinet 102. For instance, freshener compartment 152 may be recessed according to a predetermined depth along the transverse direction T. As shown in FIG. 2, the predetermined depth may vary over a length along the vertical direction V. According to some embodiments, a depth along the transverse direction T at a bottom of freshener compartment 152 is less than a depth along the transverse direction T at a top of freshener compartment. Accordingly, a rear wall of freshener compartment 152 may be angled with respect to the vertical direction V. According to another embodiment, the depth along the transverse direction T is uniform across the length of freshener compartment 152 along the vertical direction V. It should be understood that the depth of freshener compartment 152 may vary according to specific embodiments, and the disclosure is not limited to the examples described herein.

Returning briefly to FIG. 2, user interface panel 140 may include a housing 154. Hereinafter, the description of housing 154 will be made according to when user interface panel 140 is in a closed position with respect to cabinet 102. Housing 154 may include a front face 156, a pair of sidewalls 158, a top wall 160, and a bottom wall 162. Front face 156 may extend along the vertical direction V and the lateral direction L. The pair of sidewalls 158 may extend from lateral edges of front face 156 rearward (e.g., along the transverse direction T). Top wall 160 may extend from a top edge of front face 156 rearward, while bottom face 162 may extend from a bottom edge of front face 156 rearward. Accordingly, housing 154 may define a chamber 164 (e.g., bounded by front face 156, sidewalls 158, top wall 160, and bottom wall 162). Chamber 164 may face freshener compartment 152. For example, freshener compartment 152 and chamber 164 together form a cavity through which air may be circulated. Additionally or alternatively, one or more of user input devices 142 may be provided within housing 154. For instance, one or more essential controls (e.g., operational buttons, display 144, control panels, etc.) may be positioned within housing 154 to rotate together with user interface panel 140. According to some embodiments, non-essential controls may be relocated to within heating assembly cavity 146 or another part of cabinet 102. Thus, user input devices 142 may be connected through one or more physical connection means through hinges connecting user interface panel 140 to cabinet 102.

User interface panel 140 may further include a rib 166. Rib 166 may be positioned within chamber 164. Rib 166 may extend along the lateral direction L between the pair of sidewalls 158. Accordingly, rib 166 may divide chamber 164 into a first duct 168 and a second duct 170. Moreover, rib 166 may protrude rear ward along the transverse direction T from front face 156 toward freshener compartment 152. As can be seen in FIG. 2, for instance, rib 166 may contact a face of cabinet 102 above freshener compartment 152 (e.g., when user interface panel 140 is in the closed position).

First duct 168 may be defined above rib 166 (e.g., along the vertical direction V) within chamber 164. Thus, second duct 170 may be defined below rib 166 (e.g., along the vertical direction V) within chamber 164. First duct 168 may fluidly connect heating assembly cavity 146 with an ambient atmosphere (e.g., a space outside of microwave oven appliance 100, such as a kitchen atmosphere). Thus, air flow from the ambient atmosphere may be selectively circulated into heating assembly cavity 146 via first duct 168.

An air inlet 172 may be formed in housing 154. In detail, air inlet 172 may allow for fluid communication between first duct 168 and the ambient atmosphere. Accordingly, air inlet 172 may include a plurality of apertures defined through housing 154 such that air may flow into first duct 168. Air inlet 172 may be formed through top wall 160 of housing 154. Additionally or alternatively, air inlet 172 may be formed through one of the pair of sidewalls 158. According to the positioning and extension of rib 166, air entering first duct 168 via air inlet 172 may flow into heating assembly cavity 146 without flowing into second duct 170.

A secondary air inlet 173 may be formed in cabinet 102. Secondary air inlet 173 may be provided adjacent to first duct 168 (e.g., when user interface panel 140 is in the closed position). Secondary air inlet 173 may provide a passageway for the air from the ambient atmosphere to pass into heating assembly cavity 146 from first duct 168. Secondary air inlet 173 may be formed as a grate including a plurality of apertures through cabinet 102. Secondary air inlet 173 may be positioned above freshener compartment 152 (e.g., along the vertical direction V). According to at least some embodiments, secondary air inlet 173 is formed along the transverse direction T (e.g., based on a direction of air flow), however, any suitable arrangement for secondary air inlet 173 may be utilized.

Second duct 170 may be directly adjacent to freshener compartment 152. Second duct may fluidly connect freshener compartment 152 with the ambient atmosphere. For instance, second duct 170 may form a pathway through which air within heating assembly cavity 146 may flow into freshener compartment 152 before being exhausted into the ambient atmosphere.

An air outlet 174 may be formed in housing 154. In detail, air outlet 174 may allow for fluid communication between second duct 170 and the ambient atmosphere. Accordingly, air outlet 174 may include a plurality of apertures defined through housing 154 such that air may flow out of second duct 170. Air outlet 174 may be formed through bottom wall 162 of housing 154. Additionally or alternatively, air outlet 174 may be formed through one of the pair of sidewalls 158. According to the positioning and extension of rib 166, air entering second duct 170 from heating assembly cavity 146 may flow into freshener compartment 152 without flowing into first duct 168.

A freshener compartment inlet 176 may be provided between freshener compartment 152 and heating assembly cavity 146. For instance, freshener compartment inlet 176 may provide fluid communication between freshener compartment 152 and heating assembly cavity 146. Similar to secondary air inlet 173, freshener compartment inlet 176 may include a plurality of apertures defined through a rear wall of freshener compartment 152. Freshener compartment inlet 176 may be positioned at or near a top of freshener compartment 152. For instance, freshener compartment inlet 176 may be positioned adjacent to rib 166 (e.g., immediately beneath rib 166 along the vertical direction V) or at a top of second duct 170. As mentioned previously, air outlet 174 may be provided at a bottom of housing 154. Accordingly, air from heating assembly cavity 146 may enter second duct 170 at a top thereof and flow through freshener compartment 152 before exiting second duct 170 at a bottom thereof.

Freshener compartment 152 may be configured to selectively receive one or more replaceable scent cartridges. For instance, freshener compartment 152 may provide a space into which a cartridge or piece having scent-releasing capabilities may be accommodated. Accordingly, as the air from heating assembly cavity flows through second duct 170 and freshener compartment 152, a scent is picked up from the scent cartridge and released into the ambient atmosphere via air outlet 174. Additionally or alternatively, freshener compartment 152 may selectively receive an odor absorber. For instance, a packet or cartridge containing an odor absorber (e.g., baking soda, activated charcoal, hydrogen peroxide, etc.) may be positioned within freshener compartment to absorb odors produced during heating processes within appliance 100.

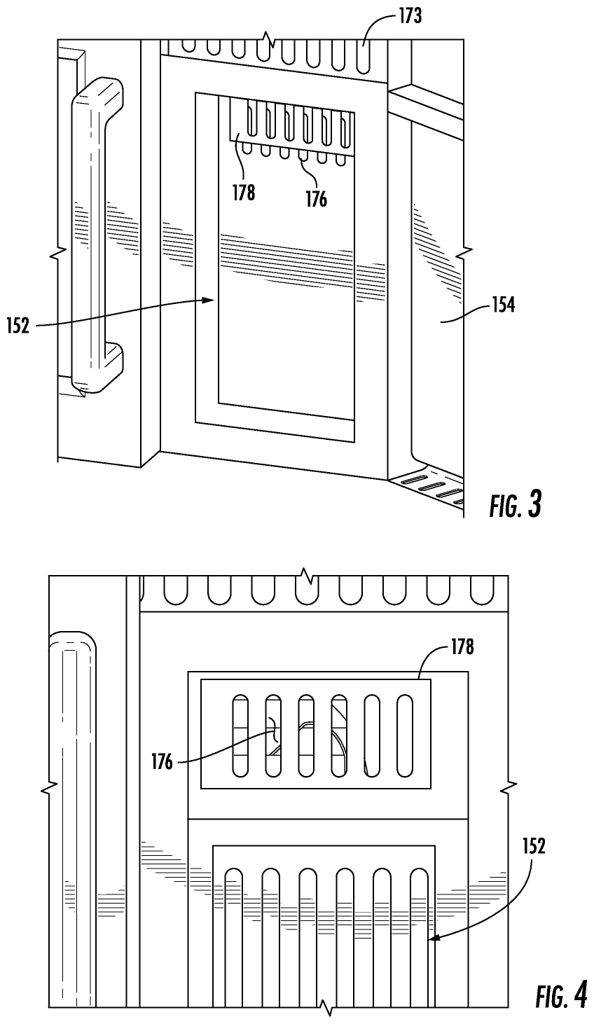

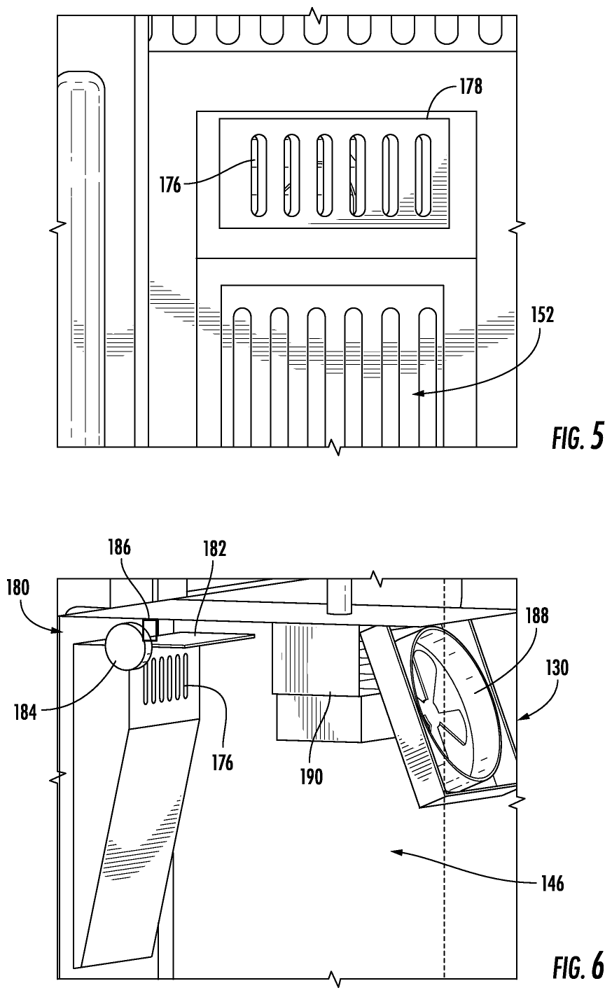

A choke slider 178 may be provided within freshener compartment 152. For instance, choke slider 178 may be movably coupled to the rear wall of freshener compartment 152 (e.g., adjacent to freshener compartment inlet 176). Choke slider 178 may be a plate shaped object slidably coupled to freshener compartment 152. Accordingly, choke slider 178 may be configured to slide along the lateral direction L with respect to freshener compartment inlet 176. Additionally or alternatively, choke slider 178 may include a plurality of apertures shaped and sized similarly to the apertures of freshener compartment inlet 176. Thus, choke slider 178 may selectively alter an air flow cross-section area of freshener compartment inlet 176 to restrict the amount of air flowing therethrough. Choke slider 178 may be manually adjusted (e.g., by a user) according to a desired air outlet. For instance, as shown in FIG. 4, choke slider 178 may be moved to a fully open position to allow a maximum amount of air to flow into freshener compartment 152. As shown in FIG. 5, choke slider 178 may be moved to a choked position such that a portion of freshener compartment inlet 176 is closed off.

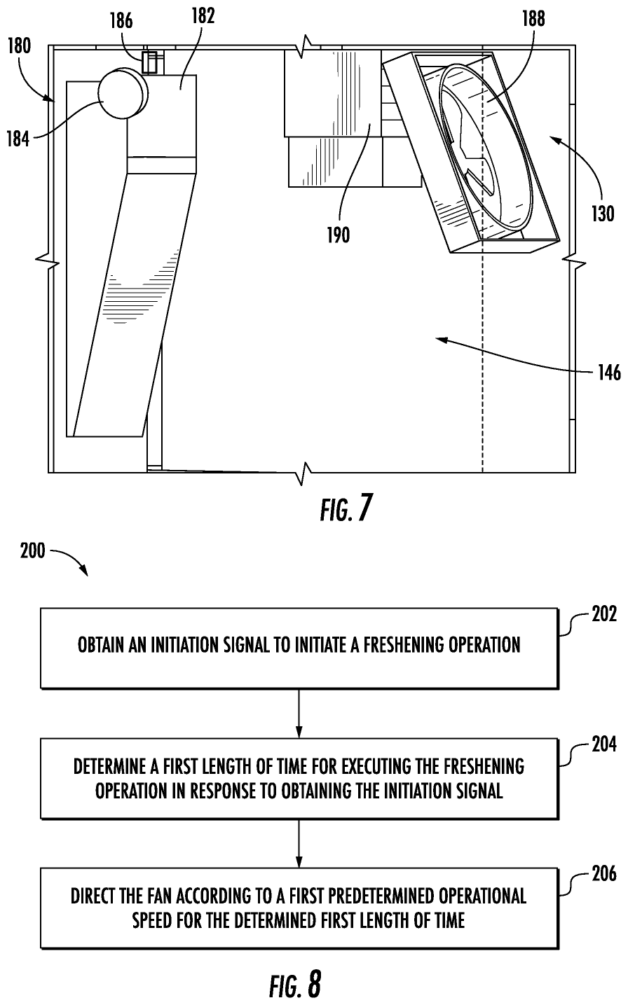

Appliance 100 may further include a damper assembly 180. Damper assembly 180 may be positioned within heating assembly cavity 146. Damper assembly 180 may include a damper door 182 and a damper motor 184. Damper door may be provided adjacent to freshener compartment inlet 176 (e.g., on an opposite side to choke slider 178). According to at least some embodiments, damper assembly 180 is provided in place of choke slider 178. Damper door 182 may be movable with respect to freshener compartment inlet 176. For instance, damper door 182 may rotate with respect to freshener compartment inlet 176 so as to selectively open and close fluid access thereto. As shown in FIG. 6, damper door 182 may be fully opened to allow a maximum amount of air to pass through freshener compartment inlet 176. Similarly, as shown in FIG. 7, damper door 182 may be fully closed to restrict air flow through freshener compartment inlet 176. Damper door 182 may be a plate shaped piece sized according to freshener compartment inlet 176 so as to fully cover freshener compartment inlet 176.

Damper motor 184 may be operationally coupled with damper door 182. Additionally or alternatively, damper motor 184 may be operationally coupled with controller 150. Accordingly, damper motor 184 may selectively rotate damper door 182 to open and close freshener compartment inlet 176. For instance, controller 150 may receive an input (e.g., via user interface controls 142, a remote input via a remote device, etc.) to open damper door 182. Damper motor 184 may then initiate a rotational movement to move damper door 184 from the closed position (FIG. 7) to the open position (FIG. 6). One or more relays or limit switches may be included to stop damper motor 184 when damper door 182 reaches the fully open or fully closed position. For instance, a limit switch 186 may be positioned within heating assembly cavity 146 to determine a position of damper door 182. Limit switch 186 may be positioned adjacent to damper motor 184, for instance. Additionally or alternatively, damper motor 184 may be controlled to position damper door 184 in a partially open or partially closed state.

Heating assembly 130 may include a fan 188 and a magnetron (or microwave energy element) 190. As best shown in FIGS. 6 and 7, heating assembly may be positioned at or near a top of heating assembly cavity 146. Fan 188 may be configured to urge a flow of air over magnetron 190. For instance, fan 188 may provide a cooling air flow to maintain magnetron 190 at a predetermined operating temperature. Additionally or alternatively, fan 188 may be operated when magnetron 190 is not operated, for instance, to provide a flow of freshener air through freshener compartment 152.

For example, fan 188 may urge the flow of air through freshener compartment 152 and out of air outlet 174 to the ambient environment. According to the illustrated exemplary embodiment, fan 188 is an axial fan positioned within heating assembly cavity 146. However, it should be appreciated that according to alternative embodiments, fan 188 may be positioned at any other suitable location and may be any other suitable fan type, such as a tangential fan, a centrifugal fan, etc.

In addition, according to an exemplary embodiment, fan 188 is a variable speed fan such that it may rotate at different rotational speeds, thereby generating different air flow rates. In this manner, the amount of air circulated through freshener compartment 152 may be regulated. Moreover, by pulsing the operation of fan 188 or throttling fan 188 between different rotational speeds, the flow of air may have a different flow velocity or may generate a different flow pattern within freshener compartment 152. Thus, by pulsating the variable speed fan or otherwise varying its speed, the flow of air may be randomized, thereby eliminating stagnant regions within freshener chamber 152 and better circulating the flow of air to provide a more even freshening profile.

Referring now to FIG. 8, a method of operating a microwave oven appliance (e.g., microwave oven appliance 100) will be described. In particular, a method of performing a freshening operation via a microwave oven appliance will be described. Although the discussion below refers to the exemplary method 200 of operating microwave oven appliance 100, one skilled in the art will appreciate that the exemplary method 200 is applicable to any suitable domestic appliance capable of performing a freshening operation. In exemplary embodiments, the various method steps as disclosed herein may be performed by controller 150 and/or a separate, dedicated controller.

At step 202, method 200 may include obtaining an initiation signal to initiate a freshening operation. The initiation signal may be obtained via a user input. For instance, the initiation signal may be input by a user via a user input control on the appliance. Additionally or alternatively, the initiation signal may be received wirelessly via a remote connected device (e.g., a smartphone). The initiation signal may include a predetermined operating speed for a fan (e.g., fan 188).

At step 204, method 200 may include determining a first length of time for executing the freshening operation in response to obtaining the initiation signal. In detail, the freshening operation may be divided into incremental operational times. For instance, the method 200 may set a baseline incremental operational time for the freshening operation. The baseline incremental operational time may be between about 8 minutes and about 15 minutes, or about 10 minutes. The first length of time for executing the freshening operation may include multiple incremental operational time inputs. For instance, the initiation signal may include a plurality of inputs for the freshening operation (e.g., multiple button presses on the user interface). As such, the first length of time may include each incremental operational time input. For one example, the incremental operational time is 10 minutes, and the initiation signal includes 3 inputs. Accordingly, the first length of time may be 30 minutes.

At step 206, method 200 may include directing the fan according to a first predetermined operational speed for the determined first length of time. The initiation signal may be directed toward a normal freshening operation. Thus, the fan (e.g., fan 188) may be directed at an initial or normal operating speed. As mentioned above, the fan may be a variable speed fan capable of operating at multiple rotational speeds to produce different levels of air flow. Thus, the normal operating speed may be a speed less than a maximum operating speed. Further to the example above, the fan may be directed at the normal operating speed for a total operational time (e.g., the first length of time).

Moreover, a total operating time of the fan may be measured and stored (e.g., on board a memory within the appliance). For instance, each minute that the fan is operational during the freshening operation may be registered therein, such that a total operational time is determined. The total operational time may be compared against a threshold (e.g., a threshold operational time). The threshold operational time may be based on a lifespan of a scent cartridge provided within the appliance (e.g., within freshener compartment 152). Accordingly, a total life of the scent cartridge may be monitored.

Upon reaching the threshold, the method 200 may emit an alert or notification. For instance, the controller may send an alert to a connected mobile device (e.g., of a user of the appliance) indicating the predicted life of the scent cartridge. The alert may include one or more suggestions. For one example, the alert notifies the user to replace the cartridge. Any suitable information may be included in the alert, included a predicted lifespan, a fan speed, etc.

According to some embodiments, the method 200 includes obtaining a boost signal to initiate a boost operation. The boost operation may include elevated operational conditions (e.g., of the fan) during the freshening operation. For instance, the boost input may include determining a second length of time (i.e., the boost operation may be performed for the second length of time). The second length of time may be longer than the first length of time. For example, when the first length of time (e.g., the first incremental operational time) is 10 minutes, the second length of time may be 15 minutes, may be 20 minutes, or greater. Additionally or alternatively, the boost operation may include a second predetermined operational speed of the fan. The second predetermined operational speed of the fan may be greater than the first predetermined operational speed. Accordingly, a higher air flow may be generated for a longer time (e.g., as compared to the normal operation), which may result in boosted scents being emitted.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A microwave oven appliance defining a vertical direction, a lateral direction, and a transverse direction, the microwave oven appliance comprising:
a cabinet, the cabinet forming a cooking cavity and a heating assembly cavity adjacent to the cooking cavity;
a door rotatably coupled to the cabinet to provide selective access to the cooking cavity;
a user interface panel coupled to the cabinet adjacent to the door;
a freshener compartment provided behind the user interface panel along the transverse direction and recessed into the heating assembly cavity, the freshener compartment being in fluid communication with the heating assembly cavity, wherein the user interface panel is movable to provide selective access to the freshener compartment; and
a heating assembly provided within the heating assembly cavity, the heating assembly comprising a fan to selectively urge air into the freshener compartment.

2. The microwave oven appliance of claim 1, wherein the user interface panel comprises:
a housing comprising a front face and a plurality of sidewalls extending from the front face to define a chamber;
a user input device provided within the housing; and
a rib extending along the lateral direction, the rib dividing the chamber into a first duct and a second duct.

3. The microwave oven appliance of claim 2, wherein the first duct fluidly connects the heating assembly cavity with an ambient atmosphere, and the second duct fluidly connects the freshener compartment with the ambient atmosphere.

4. The microwave oven appliance of claim 3, wherein the user interface panel comprises:
an air inlet formed through a top wall of the plurality of sidewalls, the air inlet providing fluid communication between the ambient atmosphere and the first duct; and
an air outlet formed through a bottom wall of the plurality of sidewalls, the air outlet providing fluid communication between the ambient atmosphere and the second duct.

5. The microwave oven appliance of claim 1, further comprising:
a freshener compartment inlet providing fluid communication between the freshener compartment and the heating assembly cavity.

6. The microwave oven appliance of claim 5, further comprising:
a choke slider slidably attached within the freshener compartment adjacent to the freshener compartment inlet, the choke slider configured to adjust an air flow area of the freshener compartment inlet.

7. The microwave oven appliance of claim 5, further comprising:
a damper assembly provided within the heating assembly cavity, the damper assembly comprising:
a damper door rotatable with respect to the freshener compartment inlet; and
a motor operably coupled with the damper door to move the damper door between an open position and a closed position.

8. The microwave oven appliance of claim 7, further comprising:
a limit switch configured to determine a position of the damper door; and
a controller operably coupled with the limit switch and the fan.

9. The microwave oven appliance of claim 1, wherein the fan is a variable speed fan.

10. The microwave oven appliance of claim 1, wherein the heating assembly further comprises:
a magnetron configured to supply microwave energy to the cooking cavity, the magnetron being positioned adjacent to the fan.

11. The microwave oven appliance of claim 1, wherein the freshener compartment is configured for receipt of one or more replaceable scent cartridges.

12. A method of operating a microwave oven appliance, the microwave oven appliance comprising a cabinet, a user interface panel coupled to the cabinet, a fan provided within the cabinet, and a freshener compartment provided behind the user interface panel, the method comprising:
obtaining an initiation signal to initiate a freshening operation;
determining a first length of time for executing the freshening operation in response to obtaining the initiation signal; and
directing the fan according to a first predetermined operational speed for the determined first length of time.

13. The method of claim 12, wherein determining the first length of time for executing the freshening operation comprises:
establishing a baseline incremental operational time for the fan;
determining a number of inputs received subsequent to obtaining the initiation signal; and
determining a total operational time of the freshening operation according to the baseline incremental operational time and the number of inputs.

14. The method of claim 12, further comprising:
obtaining a boost input signal to initiate a boost operation;
determining a second length of time for executing the boost operation; and
directing the fan according to a second predetermined operational speed for the determined second length of time, wherein the second predetermined operational speed is greater than the first predetermined operational speed.

15. The method of claim 12, wherein the user interface panel comprises:
a housing comprising a front face and a plurality of sidewalls extending from the front face to define a chamber;
a control panel provided within the housing; and
a rib dividing the chamber into a first duct and a second duct.

16. The method of claim 12, wherein the cabinet defines a cooking cavity and a heating assembly cavity adjacent to the cooking cavity, and wherein the freshener compartment is in fluid communication with the heating assembly cavity, wherein the user interface panel is movable to provide selective access to the freshener compartment.

17. The method of claim 12, wherein the freshener compartment is configured for receipt of one or more replaceable scent cartridges.

18. The method of claim 12, further comprising:
accumulating a total operational time for the freshening operation;
determining that the total operational time is equal to or greater than a predetermined threshold; and
emitting a notification, the notification comprising one or more suggestions.
